SEQUENCE LISTING (8 sequences)

SEQ ID NO: 1 - DNA, 4313 bp, Lesquerella fendleri
  CDS (574..1056, 1439..1859, 2049..2632)
gaattcggaaatgggccaagtgaaatggaaatagagcttcaatccatttagtcccactcaaaatggtgctcgaattatatttagttacgttcgaatcagacaaccaagtatttggttaataaaaaccactcgcaacaaaggaaaaacaccaagcgcgtgcgtccaacatccgacggaaggggggtaatgtggtccgaaaaccttacaaaaatctgacgtcatctacccccgaaaacgttgaatcgtcaacgggggtagttttcgaattatcttttttttaggggcagttttattaatttgctctagaaattttatgattttaattaaaaaaagaaaaagaatatttgtatatttattttttatactctttttttgtccaactatttctcttattttggcaactttaactagactagtaacttatgtcaatgtgtatggatgcatgagagtgagtatacacatgtctaaatgcatgccttatgaaagcaacgcaccacaaaacgaagacccctttacaaatacatctcatcccttagtaccctcttactactgtcccgacacaaactcaaaacaatgacatctctaaacataaagctcctttaccattacatcctaaccaaccttttcaacctctgtctcttccctttaacagcatttctcgccggaaaagcttctcacttaaccaaatccgatctcctcatgttcttatctcatctccaagacaatcttataactgtcattgtactctttactttcactatcttctgtttggttctttacattgtaaccaaacctaaacagatttatcttgtggattactcttgttaccttccaccagatcatcttaaagttagtatatcaagtgtcatggatattttctatgagttgagaaaagttgatcctttgtgtgaggtgggttgtgatgattcttctcttgagtttatgaggaaggttttagaacgttcaggtttaggtgatgagacttatgttccacttggactccatcaagtgccacctcaaaagacttttgcagcgataaaggacgagacagaacaggtaactagagccggttcaatgtttttgcatgctctatagaatttttataataatcaatataaagaaaatgacatattaattgataaaattgttataaaataaagttttgatccccttaaaaatctagaagcttataaattttgaaaatttaggtcgaaaaatgtttaatatttataattttttaaaaaaaattagatgtaaaatgtcgtaaaaattgtgatactatagatgaatcgtctactttttgaacatgtcctggggtgatagaagcggaccaagcctatgcttgtggttgttagattgtttattctttttatttagtttgtatcctttattttttctgtcgcttgtggtcttaaaatgtataggaccggctctgcaggtaatcaaaggtgcacttgagaatctattcgagaacacaaaagtaaaccctagagagattggtatacttgtgataaactcaagcatgtttaatccaacaccttctctatcagccatggttattaatactttcaaactccgaagtaacatcaaaagctttaatcttggaggaatgggttgtagtgctggtgtaatcgccattgatctagctaaagacttgttgcatgttcataaaaacacttatgctcttgtaataagcactgagaacatcaccataaccgcttatgctggcgaaaatcgatccatgaatgttagtaattgcttgttccgaataggcggggccgcgattttgctctctaataagccaagagataagagaaggtctaagtacaagctagctcacactgttcgaacacatacaggtattgaatctagatgattacattgcgaagaaaatgttttattcataagcataaggtgccaaataggatacaattttgctatcatataatatcgtcatcatatcagaattatatcgaatatgatatcttataacatatgattttctatatgatgtgattctctaaatcttctagtatgttctacaacaggagctgatgacatgtcttatagatgtgtgcaacaagaagaagatgagatgggtaaagtaggagttcgtctctcaaaggacataactactgtcgcgggcacagcagttaagaagaacatatcaacattaggtccactgattcttcctttaagcgaaaagcttctttatttcgtttccttcatcgcgaagaaacttttgaaggagaagatcaagaactattacgtcccggatcttaagctagctatcaatcatttttgtatccatgctggtggaagaggtgtgttagatgtgttggaaaagaacttaaggctatcaccaattgatgtagaagcatctagatcgactttacatagatttgggaatacttcttcgagttcgatttggtatgagttggcttatattgaagctaaaggaaggatgaagaaaggtgataaagcttggcaaattgctttagggtcagggtttaagtgtaatagtgctgtttgggttgctttacgtaatgttaaagcttcggctaatagtccttgggaacattgtattgataggtatcctgttcctgatacttgtgtagaaaatggtaagtcttagttaatgtatcttagtgtcaacgtttcttttttttttttagttttttttttgatgttgaagatgttctcttttcgttataaataagaaataaaagtttttttaggaggactgccatttaagtttaccaatcttgtctatactgccatttagcttcagcgtgaacattaatactacggacggatatgttataggcttgtcattgtccgcgaaaaaagatcaatgtactcctgtgtttacagatatttgcgattgaacctgggtaaacagtccgttttgattgttatggacaatcatgtactacacgcgttatccgtattgttcacagaagatcaaaacaaaccattttatccatattaaatcgtaaatatctgtaaacacaaagatacgcggtctatttcgcagaaagtagcacgcttgatcgaatccgtatatggaaattaaaaaacgtctttgaaaatatcaatttcataatttcaataaagagaagttaacttaaccggtagtttcaagaaatttccaatagaaattaaccggcagtcatcatttttattgggccttctgtggactatagaatcaggccctttattgtaacttgatatagcctttagaatcaggcaggccgtttattcataaatcgaaaatcttgggataattaagaatcaacaacttggaatcgggaccagtcgcagcaaattgatgtcgtaacgtgtcacttggcatgttttagttaggaccatattcgaaaaagtaaatgggtcatgttgccataaaacaaaaaatgctgattccaaacgtaaattgctaaactcgatgtaataaaggttagattttacgaacctcaaacccccatttttttactttgtttatctccaatcccacggtgtaagaacaacaagaacatgatgctttaaataatagaagacaaactcataaaattgaaataaatagtcatcatactagcagattaagatccgaattttacaaccaaaaacaaataaatgccttttttttttttttttaattcggatcttcttattcatattcttattaataatttttattaaaagatactaagaaatcgtgcaattacgtttctacgatgataaatcaaacccatcgtcgcagtagtaaaacccaaagttattgatcgcagctgaaaagagagttactctgtgatgctgcctcagttgcaagatacggcatgttgtgaaaggaacgatcaagacaagacacaacattgttgatctcgtcttcgtcttcatcgtcatcgcttttggtatgcatcaattcatctgctcggtgctttagagtctcaaacattagctcaggctcatgctgcattgctcgaagaacatcagcagaggactgaccaggcatggctcgggtaactgcaaagctatgtggtccatcgctctgcgagacacggacaatgcttggcccaccaaacaaattatggactccaccgagcgcctcctcataagctcctccaagaaacattcccagaaagtatcttcctccgcagttgtcaatctcgtgcaatggcatggtggattctccgcctatgaacttgttgatctttccatcgctatcgcaagttaaatcagataagatcccacgagttcccggcctttgatcgagcttatggataggaactataggaaataactgatccgaattc

SEQ ID NO: 2 - protein, 496 aa, Lesquerella fendleri
MTSLNIKLLYHYILTNLFNLCLFPLTAFLAGKASHLTKSDLLMFLSHLQDNLITVIVLFTFTIFCLVLYIVTKPKQIYLVDYSCYLPPDHLKVSISSVMDIFYELRKVDPLCEVGCDDSSLEFMRKVLERSGLGDETYVPLGLHQVPPQKTFAAIKDETEQVIKGALENLFENTKVNPREIGILVINSSMFNPTPSLSAMVINTFKLRSNIKSFNLGGMGCSAGVIAIDLAKDLLHVHKNTYALVISTENITITAYAGENRSMNVSNCLFRIGGAAILLSNKPRDKRRSKYKLAHTVRTHTGADDMSYRCVQQEEDEMGKVGVRLSKDITTVAGTAVKKNISTLGPLILPLSEKLLYFVSFIAKKLLKEKIKNYYVPDLKLAINHFCIHAGGRGVLDVLEKNLRLSPIDVEASRSTLHRFGNTSSSSIWYELAYIEAKGRMKKGDKAWQIALGSGFKCNSAVWVALRNVKASANSPWEHCIDRYPVPDTCVENGKS

SEQ ID NO: 3 - DNA, 23 bp, Artificial Sequence
  FAE1 Primer
ccgagctcaaagaggatacatac

SEQ ID NO: 4 - DNA, 29 bp, Artificial Sequence
  FAE1 Primer
gatactcgagaacgttggcactcagatac

SEQ ID NO: 5 - DNA, 29 bp, Artificial Sequence
  PCR Primer
cgcaagcttgaattcggaaatgggccaag

SEQ ID NO: 6 - DNA, 29 bp, Artificial Sequence
  PCR Primer
cgcgtcgactgttttgagtttgtgtcggg

SEQ ID NO: 7 - protein, 506 aa, Arabidopsis thaliana
MTSVNVKLLYRYVLTNFFNLCLFPLTAFLAGKASRLTINDLHNFLSYLQHNLITVTLLFAFTVFGLVLYIVTRPNPVYLVDYSCYLPPPHLKVSVSKVMDIFYQIRKADTSSRNVACDDPSSLDFLRKIQERSGLGDETYSPEGLIHVPPRKTFAASREETEKVIIGALENLFENTKVNPREIGILVVNSSMFNPTPSLSAMVVNTFKLRSNIKSFNLGGMGCSAGVIAIDLAKDLLHVHKNTYALVVSTENITQGIYAGENRSMMVSNCLFRVGGAAILLSNKSGDRRRSKYKLVHTVRTHTGADDKSFRCVQQEDDESGKIGVCLSKDITNVAGTTLTKNIATLGPLILPLSEKFLFFATFVAKKLLKDKIKHYYVPDFKLAVDHFCIHAGGRAVIDELEKNLGLSPIDVEASRSTLHRFGNTSSSSIWYELAYIEAKGRMKKGNKAWQIALGSGFKCNSAVWVALRNVKASANSPWQHCIDRYPVKIDSDLSKSKTHVQNGRS

SEQ ID NO: 8 - protein, 505 aa, Brassica napus
MTSINVKLLYHYVITNLFNLCFFPLTAIVAGKAYLTIDDLHHLYYSYLQHNLITIAPLLAFTVFGSVLYIATRPKPVYLVEYSCYLPPTHCRSSISKVMDIFFQVRKADPSRNGTCDDSSWLDFLRKIQERSGLGDETHGPEGLLQVPPRKTFARAREETEQVIIGALENLFKNTNVNPKDIGILVVNSSMFNPTPSLSAMVVNTFKLRSNVRSFNLGGMGCSAGVIAIDLAKDLLHVHKNTYALVVSTENITYNIYAGDNRSMMVSNCLFRVGGAAILLSNKPRDRRRSKYELVHTVRTHTGADDKSFRCVQQGDDENGQTGVSLSKDITDVAGRTVKKNIATLGPLILPLSEKLLFFVTFMGKKLFKDEIKHYYVPDFKLAIDHFCIHAGGKAVIDVLEKNLGLAPIDVEASRSTLHRFGNTSSSSIWYELAYIEPKGRMKKGNKVWQIALGSGFKCNSAVWVALNNVKASTNSPWEHCIDRYPVKIDSDSGKSETRVPNGRS

(19) United States
(12) Patent Application Publication
(10) Pub. No.: US 2004/0049806 A1
(43) Pub. Date: Mar. 11, 2004

(54) Nucleic acid encoding a plant very long chain fatty acid biosynthetic enzyme

(76) Inventors: Ljerka Kunst, Vancouver (CA); Mark Andrew Smith, Vancouver (CA); Hangsik Moon, Ames, IA (US)

Correspondence Address: MORGAN LEWIS & BOCKIUS LLP, 1111 PENNSYLVANIA AVENUE NW, WASHINGTON, DC, 20004, US

(21) Appl. No.: 10/276,977

(22) PCT Filed: May 24, 2001
(86) PCT No.: PCT/IB01/01140

(51) Int. Cl. A01H 1/00; C12N 15/82; C12Q 1/68; C07H 21/04; C12N 9/10; C12N 5/04

(52) U.S. Cl. 800/281; 435/6; 435/69.1; 435/320.1; 435/193; 435/419; 536/23.2

(57) ABSTRACT

A genomic DNA sequence encoding a condensing enzyme involved in very long chain fatty acid production in plants is provided. Applications for the isolated nucleic acid sequence to include the expression of the condensing enzyme of the present invention in seeds, which would allow the production of crop plants, which are capable of synthesizing hydroxylated very long chain fatty acids in seed oil for industrial applications.

This application claims priority from U.S. Provisional Patent Application No. 60/206,789, which was filed May 24, 2000.

### FIELD OF THE INVENTION

This invention relates to the isolation of a genomic DNA sequence encoding a condensing enzyme involved in very long chain fatty acid production in plants and its uses.

### BACKGROUND

Living organisms synthesize a vast array of different fatty acids which are incorporated into complex lipids. These complex lipids represent both major structural component membranes, and are a major storage product in both plants and animals. Very long chain fatty acids (VLCFAs, chain length C20 or longer) are synthesized in the epidermal cells where they are either directly incorporated into waxes, or serve as precursors for other aliphatic hydrocarbons found in waxes, including alkanes, primary and secondary alcohols, ketones aldehydes and acyl-esters. VLCFAs also accumulate in the seed oil of some plant species, where they are incorporated into triacylglycerols (TAGs), as in the Brassicaceae, or into wax esters, as in jojoba. These seed VLCFAs include the agronomically important erucic acid (C22:1), used in the production of lubricants, nylon, cosmetics, pharmaceuticals and plasticizers.

VLCFAs are synthesized by a microsomal fatty acid elongation (FAE) system which involves four enzymatic reactions: (1) condensation of malonyl-CoA with a long chain acyl-CoA, (2) reduction to -hydroxyacyl-CoA, (3) dehydration to an enoyl-CoA and (4) reduction of the enoyl-CoA, resulting in the elongated acyl-CoA by two carbons. The condensing enzyme catalyzing reaction (1) is the key activity of the FAE system. It is the rate-limiting enzyme of the VLCFA biosynthetic pathway, which controls the amount of VLCFAs produced. In addition, the condensing enzyme determines the ultimate VLCFA acyl chain length, and thus their use.

### SUMMARY OF THE INVENTION

The present invention consists of a DNA sequence encoding a condensing enzyme involved in VLCFA biosynthesis. Such a DNA fragment is desirable for use in genetic engineering projects aimed at increasing the chain length of fatty acids in seed oils. In addition, expression of this sequence in the epidermis can be used for altering the composition and accumulation of cuticular and epicuticular waxes.

### BRIEF DESCRIPTION OF THE DRAWINGS

FIG. 1 shows DNA sequence of the LfKCS3 genomic clone. The deduced amino acid sequence in shown below the nucleotide sequence of corresponding exons. Intron sequences are shown in bold and italics.

FIG. 2 shows sequence similarity among the Brassicaceae condensing enzymes along their entire length (FIG. 2).

### DETAILED DESCRIPTION

The present invention provides an isolated genonic DNA sequence encoding a condensing enzyme involved in very long chain fatty acid production in plants. Because condensing enzymes are pivotal enzymes in the synthesis of very long chain fatty acids (VLCFA), controlling levels of accumulation of VLCFAs and their acyl chain length (Millar and Kunst, 1997), are useful for biotechnology. For instance, the accumulation of VLCFAs in tobacco seed expressing FAE1 from Arabidopsis (Millar and Kunst, 1997) indicates that VLCFAs can be produced in plant species that currently do not synthesize VLCFAs. The availability of LfKCS3 condensing enzyme may be especially useful, because it is capable of efficiently elongating hydroxy fatty acids. Thus, the expression of the LfKCS3 condensing enzyme in seeds should allow the production of crop plants capable of synthesizing hydroxylated VLCFAs in seed oil for industrial applications.

The methods employed in the isolation of the nucleic acid sequence of the present invention and the uses thereof are discussed in the following non-limiting examples:

Screening of the Genomic DNA Library:

A Lesquerella fendleri genomic DNA library was obtained from Dr. Chris Somerville of the Carnegie Institution of Washington, Stanford, Calif. The genomic library was plated on E. coli LE392 (Promega) and about 150,000 clones were screened using Arabidopsis FAE1 as a probe. The probe was prepared by PCR using pGEM7-FAE1 (Millar and Kunst, 1997) as a template with FAE1 upstream primer, 5′-CCGAGCTCAAAGAGGATACATAC-3′ and FAE1 downstream primer, 5′-GATACTCGAGAACGTTGGCACTCAGATAC-3′. PCR was performed in a 10 μl reaction containing 10 ng of the template, 2 mM MgCl₂, 1.1 μM of each primer, 100 μM of (dCTP+dGTP+dTTP) mix, 50 μCi of [α-32P]dATP, 1×PCR buffer and 2.5 units of Taq DNA polymerase (Life Technologies). Amplification conditions were: 2 min of initial denaturation at 94° C., 30 cycles of 94° C. for 15 sec, 55° C. for 30 sec, 72° C. for 1 min and 40 sec, followed by a final extension at 72° C. for 7 min. The amplified hot probe was purified by QIAqiuck PCR Purification Kit (Qiagen) and denatured by boiling before adding to the hybridization solution. Hybridization took place overnight at 65° C. in a solution containing 6×SSC (1×SSC=0.15 M NaCl, 0.015 M Na-citrate, pH 7.0), 20 mM NaH₂PO₄. 0.4% SDS, 5×Denhardt's solution [0.1% (w/v) of Ficoll (Type 400, Pharmacia), 0.1% (w/v) of polyvinylpyrrolidone, and 0.1% (w/v) of bovine serum albumin (Fraction V, Sigma)], and 50 μg/ml sonicated, denatured salmon sperm DNA (Sigma) and washing was performed three times for 20 min each in 2×SSC, 0.5% (w/v) SDS at 65° C.

Construction of Plasmids (Refer to Table 1):

From tertiary screening, nine positive clones were purified from the Lesquerella fendleri genomic library. The phage DNA from those nine clones was extracted and purified using QIAGEN Lambda Mini Kit (Qiagen) according to the manufacturer's protocol. One of them was digested with EcoRI and a 4.3 kb fragment was subcloned into the pGEM-7Zf(+) vector (Promega) cut with EcoRI, resulting in the vector pMHS15. The whole insert was sequenced with ABI automatic 373 DNA sequencer using fluorescent dye terminators. The upstream region of the genomic DNA was amplified using the high fidelity Pfu polymerase (Stratagene) with a forward primer 5′-CGCAAGCTTGAATTCGGAAATGGGCCAAGT3′ and a reverse primer 5′-CGCGTCGACTGTTTTGAGTTTGTGTCGGG-3′. The amplified 573 bp promoter was inserted upstream of the GUS gene in pBI101 (Clontech) cut with HindIII and SalI, resulting in the vector pLfKCS3-GUS. The fragment containing the promoter and the coding sequence was removed from pMHS15 by digestion with EcoRI and HpaI and the insert fragment was ligated to pRD400 cut with EcoRI and SmaI, resulting in the vector pLfKCS3. As a comparison, another construct was made with LFAH12 promoter (Broun et al., 1998) and the coding sequence, which was named pLFAH12-LfKCS3.

TABLE 1
Plasmids

| Name | Promoter | Coding sequence(s) |
|---|---|---|
| pMHS15 | 4313 bp EcoRI fragment in pGEM-7Zf(+) | |
| pLfKCS3-GUS | LfKCS3 (573 bp) | β-glucuronidase |
| pLfKCS3 | LfKCS3 (573 bp) | LfKCS3 ORF (2062 bp) |
| P1FAH12-LfKCS3 | LFAH12 (2200 bp) | LfKCS3 ORF (2062 bp) |

All the plasmids shown in Table 1, pLfKCS3-GUS, pLfKCS3, and pLFAH12-LfKCS3, were introduced into Agrobacterium tumefaciens strain GV3101 (pMP90; Koncz and Schell, 1986) by heat-shock and selected for resistance to kanamycin (50 μg/mL). They were then used to tansform Arabidopsis (Columbia wild type) and/or fad2/fae1 double mutant by floral dip method (Clough and Bent, 1998). The fad2/fae1 double mutant is characterized by a very high level (>80%) of oleic acid (18:1) in its seed oil due to deficiency in the activities of both cytoplasmic oleate Δ12 desaturase and the condensing enzyme, FAE1. Screening for transformed seed was done on 50 μg/mL kanamycin as described previously (Katavic et al., 1994).

Analysis of Fatty Acid Composition:

To determine the fatty acid composition of the tissues, fatty acid methyl esters were prepared by refluxing the samples in 2 ml of 1N methanolic-HCl for 90 min at 80° C. After cooling 2 ml of 0.9% NaCl solution and 200 μl of hexane were added and the mixture was vortexed vigorously. The fatty acid methyl esters in the hexane layer were analyzed by gas chromatography.

GUS Assay:

GUS assay was performed by immersing tissues in GUS histochemical staining solution (Jefferson, 1989) for 4 to 7 hours at 37° C. The assay solution was composed of 50 mM sodium phosphate, pH 7.0, 0.5 mM potassium ferricyanide, 0.5 mM potassium ferrocyanide, 10 mM EDTA, 0.05%(w/v) triton X-100, and 0.35 mg/ml 5-bromo-4chloro-3-indolyl-β-D-glucuronide (X-Gluc). Following staining the blue-stained samples were fixed in 70% ethanol.

Isolation and Characteristics of the LfKCS3 Gene:

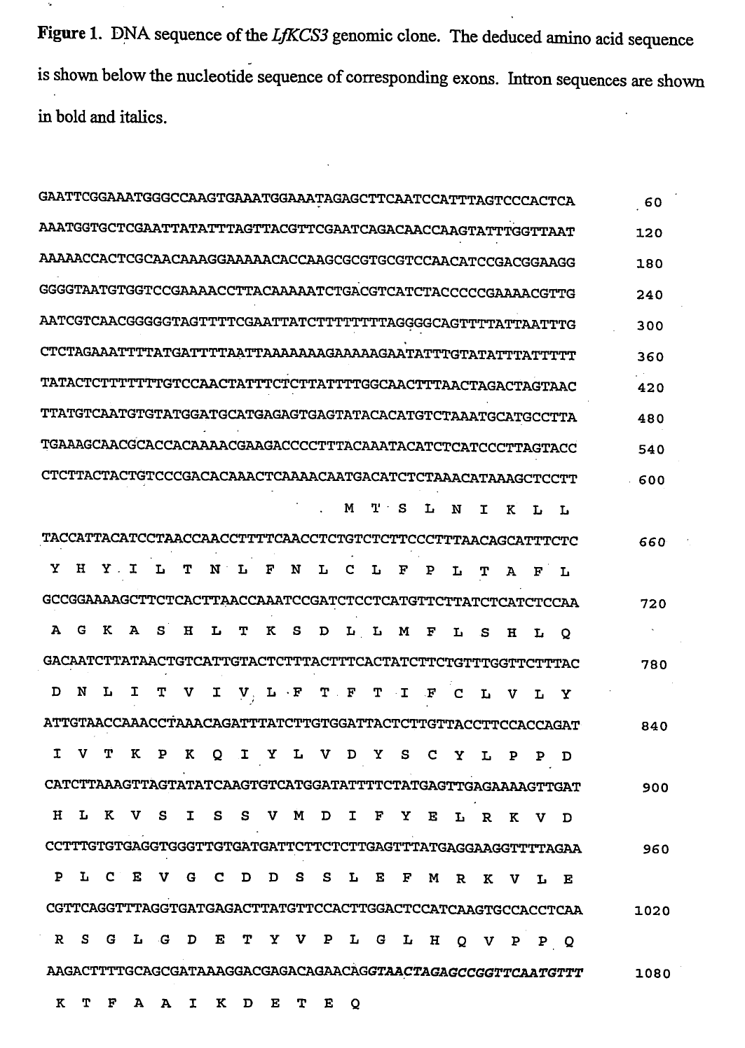

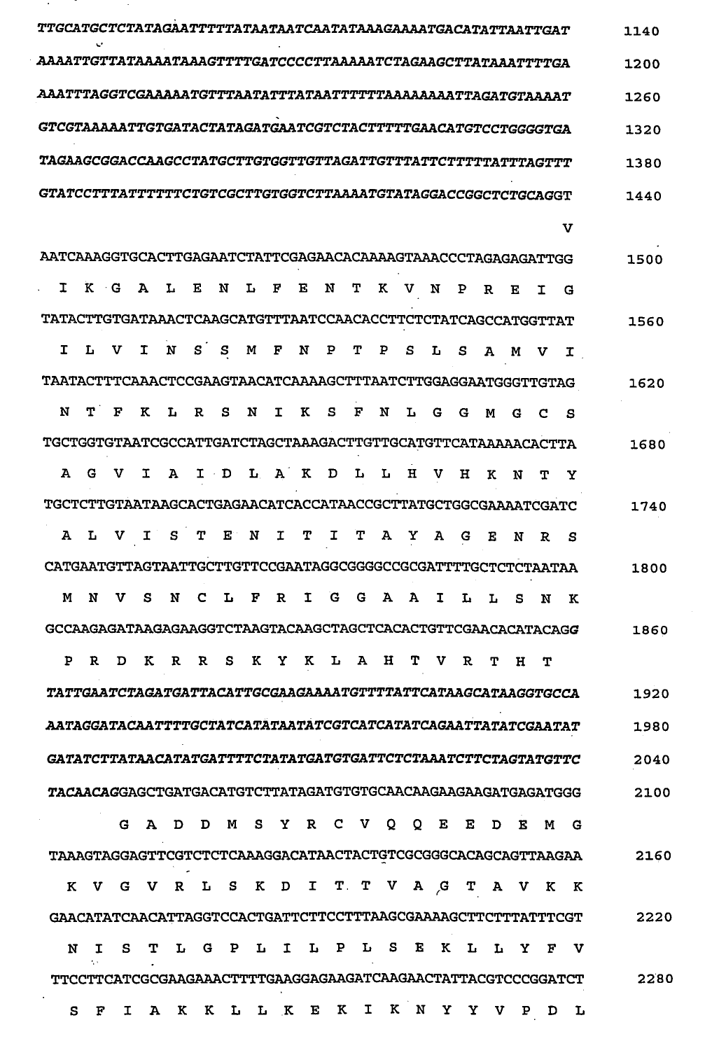

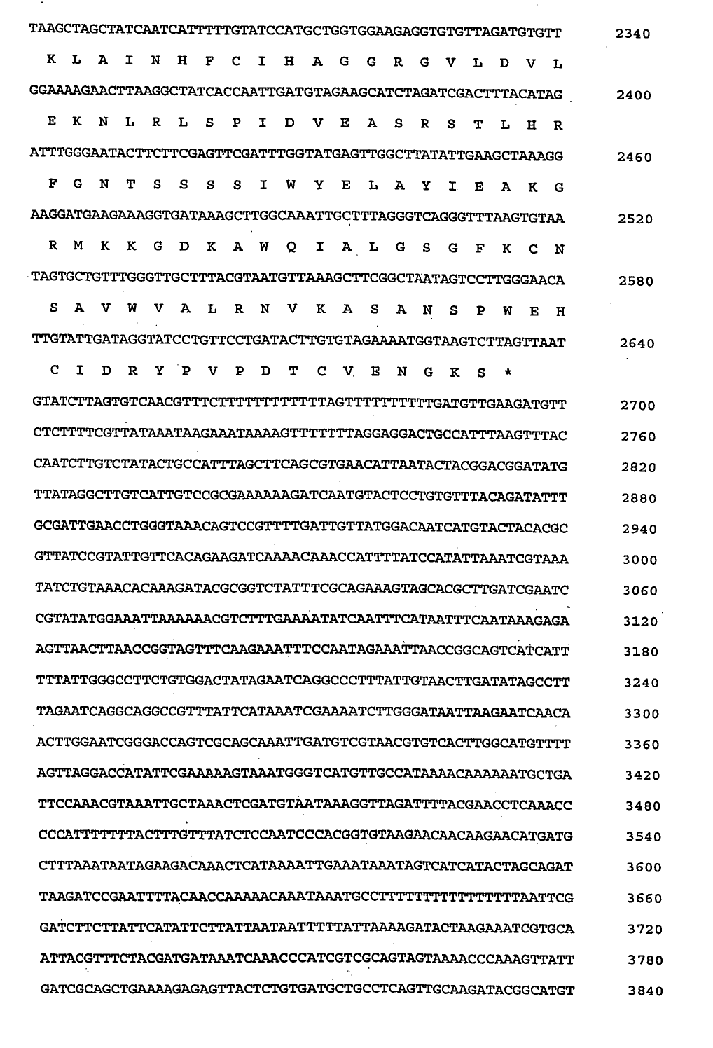

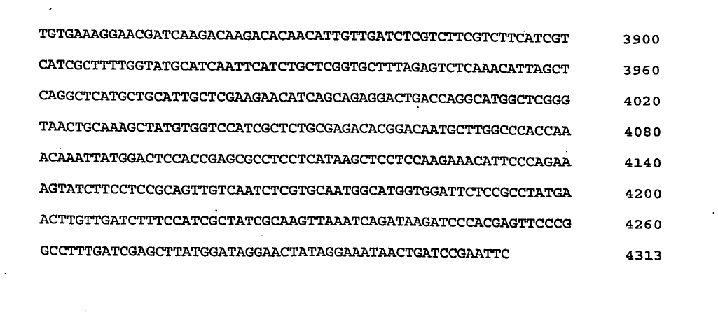

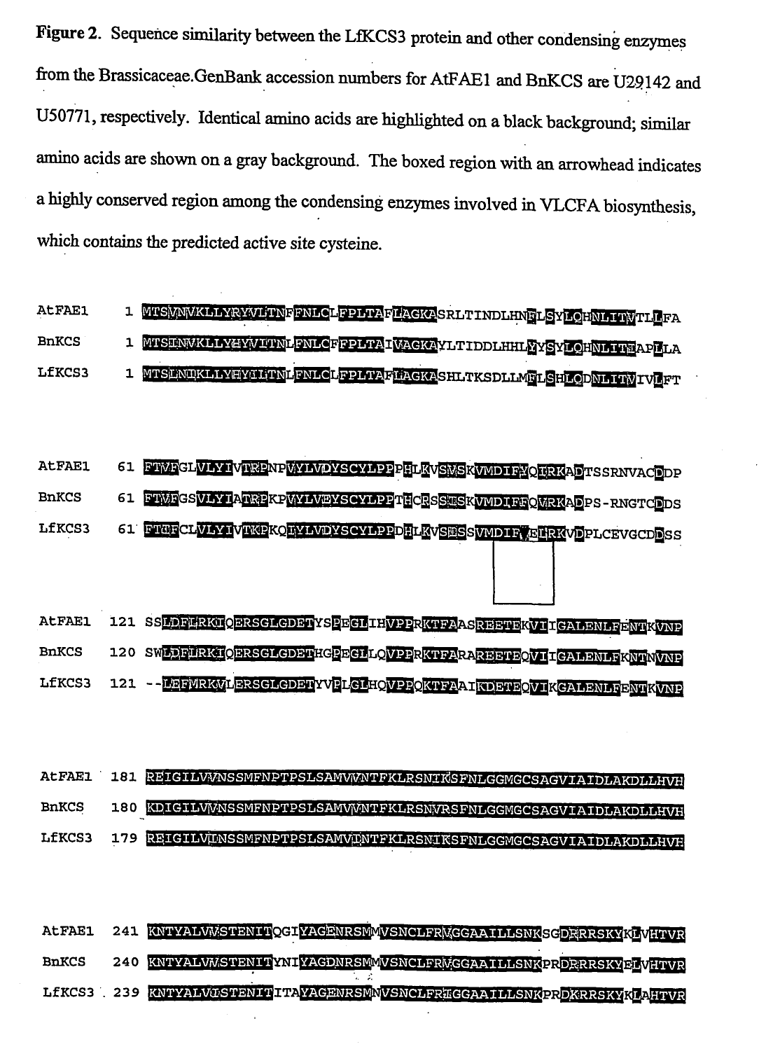

A genomic clone of a putative condensing enzyme was isolated using the Arabidopsis FAE1 (James et al., 1995) to probe filters of a genomic library of Lesquerella fendleri. The EcoRI fragment subcloned into the plasmid pMHS15 was fully sequenced and a 4313 bp consensus sequence was assembled from individual sequence fragments using GCG program (Edelman et al., 1994). The sequence included 573 bp of 5′ flaking region, a 2062 bp coding region, and an 1678 bp 3′ flanking sequence (FIG. 1). A sequence comparison between the 4313 bp genomic DNA and the Arabidopsis cDNA made using the BCM Search Launcher: Multiple Sequence Alignments (Smith et al., 1996) revealed two introns in the L. fendleri genomic DNA. Then the deduced amino acid sequence was obtained after removing the two introns by aid of a translation tool, “Translate” (http://www.expasy.ch/tools/dna.html). The amino acid sequence indicates that the gene, designated LfKCS3, encodes a polypeptide of 496 amino acids, and an estimated molecular mass of the LfKCS3 protein is 55.3 kD. Amino acid sequence comparisons, using the BCM Search Launcher: Pairwise Sequence Alignments (Smith et al., 1996), show that LfKCS3 shares high sequence identity, ranging from 49.9% to 80.2%, with VLCFA condensing enzymes, including Arabidopsis FAE1 (James et al., 1995), Brassica napus KCS (Roscoe et al., 1996: GenBank accession number U50771), CUT1 (Miller et al.; 1999), and jojoba KCS (Lassner et al., 1996). Multiple sequence alignments reveal remarkable similarity among the Brassicaceae condensing enzymes along their entire length (FIG. 2).

Expression Studies of LfKCS3 in Plants:

In order to determine the LfKCS3 expression pattern a 573 bp upstream fragment from the LfKCS3 genomic clone was translationally fused to the uidA reporter gene encoding β-glucuronidase (GUS), and introduced into Arabidopsis by floral dip method (Clough and Bent, 1998). LfKCS3 expression pattern was determined for more than 30 independent primary transgenic plants using GUS histochemical assays on leaves, stems, inflorescences, roots, and siliques at different stages of development. GUS staining was observed exclusively in the embryos. No GUS expression was detected in other tissues. Thus, the Arabidopsis LfKCS3 promoter is regulated in a tissue specific manner.

### References

Clough, S. J. and Bent, A. F. (1998) Floral dip: a simplified method for Agrobacterium-mediated transformation of Arabdiopsis thaliana. Plant J. 16, 735-743.

Broun, P., Boddupanli, S., and Somerville, C. (1998) A bifunctional oleate 12-hydroxylase:desaturase from Lesquerella fendleri. Plant J. 13, 201-210

Doyle, J. J., and Doyle, J. L. (1990) Isolation of plant DNA from fresh tissue. Focus 12, 13-15

Edelman, L, Olson, D., and Devereux, J. (1994) Wisconsin Sequence Analysis Package, version 8.0, Genetic Computer Group, Inc., University Research Park, 575 Science Drive, Madison, Wis., 53711, USA

James, D. W., Lim, E., Keller, J., Plooy, I., Ralston, E., and Dooner, H. K. (1995) Directed tagging of the Arabidopsis FATTY ACID ELONGATION1 (FAE1) gene with the maize transposon activator. Plant Cell 7, 309-319

Jefferson, R. A. (1987) Assaying chimeric genes in plants: The GUS gene fusion system. Plant Mol. Bol. Rep. 5, 387-405

Katavic, V., Haughn, G. W., Reed, D., Martin, M., and Kunst, L. (1994) In planta transformation of Arabidopsis thaliana Mol.Gen.Genet 245, 363-370.

Koetsier, P. A., Schorr, J., and Doerfler, W. (1993) A rapid optimized protocol for downward alkaline Southern blotting of DNA. BioTechniques 15, 260-262

Koncz, C. and Schell, J. (1986) The promoter of T_{L}-DNA gene 5 controls the tissue-specific expression of chimaeric genes carried by a novel type of Agrobacterium binary vector. Mol. Gen. Genet 204, 383-396.

Lassner, M. W., Lardizabal, K., and Metz, J. G. (1996) A jojoba β-ketoacyl-CoA synthase cDNA complements the canola fatty acid elongation mutation in transgenic plants. Plant Cell 8, 281-292

Millar, A. A., and Kunst, L. (1997) Very-long-chain fatty acid biosynthesis is controlled through the expression and specificity of the condensing enzyme. Plant J. 12, 121-131

Millar, A. A., Clemens, S., Zachgo, S., Giblin, E. M., Taylor, D. C., and Kunst, L. (1999) CUT1, an Arabidopsis gene required for cuticular wax biosynthesis and pollen fertility, encodes a very-long-chain fatty acid condensing enzyme. Plant Cell 11, 825-838

Smith, R. F., Wiese, B. A., Wojzynski, M. K., Davison, D. B., Worley, K. C. (1996) Search-Launcher—An integrated interface to molecular biology database search and analysis services available on the world wide web. Genome Res. 6, 454-462

van de Loo, F. J., Broun, P., Tumer, S., and Somerville, C. (1995) An oleate 12-hydroxylase from Ricinus communis L. is a fatty acyl desaturase homolog. Proc. Natl. Acad. Sci. USA 92,6743-6747

What we claim is:

1. An isolated nucleic acid fragment comprising a nucleic acid sequence encoding a condensing enzyme involved in very long chain fatty acid production in plants.

2. An isolated nucleic acid fragment according to claim 1, wherein said sequence is defined by the nucleic acid sequence of SEQ. ID. NO. 1.

3. An isolated nucleic acid fragment according to claim 1, wherein said sequence has a sequence identity of 95% or greater to the nucleic acid sequence of SEQ. ID. NO. 1.

4. An isolated nucleic acid fragment according to claim 1, wherein said sequence has a sequence identity of 85% or greater to the nucleic acid sequence of SEQ. ID. NO. 1.

5. An isolated nucleic acid fragment according to claim 1, wherein said sequence has a sequence identity of 65% or greater to the nucleic acid sequence of SEQ. ID. NO. 1.

6. An isolated nucleic acid fragment comprising a nucleic acid sequence encoding an enzyme for enhancing expression of endogenous and foreign genes in seeds for the enhanced production of seed oils.

7. An isolated nucleic acid fragment according to claim 1, wherein said sequence promotes expression of genes, which enhance seed production of long chain fatty acids in plants

8. An isolated nucleic acid fragment according to claim 3, wherein said genes enhance the nutritive value of seed production in plants.

9. An isolated nucleic acid fragment comprising a nucleic acid sequence encoding a condensing enzyme, which promotes production of very long chain fatty acids production in plants, which naturally do not synthesize very long chain fatty acids.
